(19) United States
(12) Patent Application Publication
(10) Pub. No.: US 2011/0287472 A1
(43) Pub. Date: Nov. 24, 2011

(54) MODULAR SYSTEM OF FUNCTIONAL UNITS FOR MIXING, PROCESSING AND/OR SEPARATING SAMPLES FOR USE IN BIOLOGICAL/MEDICAL RESEARCH AND FOR DIAGNOSTICS

(75) Inventors: Jan-Michael Heinrich, Leipzig (DE); Christoph Mohr, Leipzig (DE)

(73) Assignee: UNIVERSITAT LEIPZIG, Leipzig (DE)

(21) Appl. No.: 13/122,778

(22) PCT Filed: Oct. 6, 2009
(86) PCT No.: PCT/DE09/01411

§ 371 Date: Aug. 10, 2011

(30) Foreign Application Priority Data
Oct. 6, 2008 (DE) 10 2008 050 750.4

(51) Int. Cl. C12Q 1/02; G01N 1/18; C12M 1/00; B01J 8/00

(52) U.S. Cl. 435/29; 422/187; 436/178; 435/283.1

(57) ABSTRACT

Mixing, physical and/or chemical reactions and separation are basic method steps in biological research and diagnosis. In research in particular, there is a need for problem-specific laboratory systems, but these are not commercially available because of small batch sizes and because of the specific problem involved. In the absence of these systems, existing vessels, filters, centrifuges, etc., have to be improvised in order to solve the problem.

The aim of the invention is to make available a system of functional units that can in each case be plugged together depending on the methodological problem.

The system according to the invention comprises freely combinable functional units and consists of mixing cylinders, separating devices and a flow-regulating connection unit, preferably formed as a vacuum unit, which is designed such that they are connected, preferably via simple plug connections, in any desired sequence to as many functional units as necessary in order to form an inherently closed sample-processing system.

### CROSS-REFERENCE TO RELATED APPLICATIONS

This application is the United States national phase under 35 U.S.C. §371 of PCT International Patent Application No. PCT/DE2009/001411, filed on Oct. 6, 2009, and claiming priority to German Application No. 10 2008 050 750.4, filed on Oct. 6, 2008.

### BACKGROUND OF THE INVENTION

1. Field of the Invention

Embodiments relate to the field of sample separation for medical and biological diagnosis.

2. Background of the Related Art

Mixing, physical and/or chemical reactions and separation are basic method steps in biological research and diagnosis. In research in particular, there is a need for problem-specific laboratory systems, but these are not commercially available because of small batch sizes and because of the specific problem involved. In the absence of these systems, existing vessels, filters, centrifuges, etc., have to be improvised in order to solve the problem.

In practice, when processing small batches of samples, it has also hitherto been necessary to perform several successive work steps involving decanting, pipetting, filtering, centrifuging, etc., of the samples, which requires the samples to be successively transferred by hand between different vessels and which, in addition to the extensive labour involved, also requires a high level of output in terms of equipment, since only some of the work steps are generally carried out in each vessel. In addition to the “human” factor, the various laboratory set-ups found in different laboratories (particularly as regards vessels, pipettes, filters, etc.) also often mean that, when following the same operating instructions, different results are obtained in the individual laboratories, such that complicated optimisation procedures are then required for the reproducibility and/or standardisation of the results in the individual laboratories.

### BRIEF SUMMARY OF THE INVENTION

The aim of the invention is therefore to make available a system of functional units that can in each case be combined, preferably plugged together, depending on the methodological problem, and that permit simple and reproducible mixing, physical and/or chemical reactions and separation of sample media.

Surprisingly, a modular system (hereinafter also called “system” or “system according to the invention”) consisting of one or more mixing cylinders, one or more separating devices and a flow-regulating connection unit, which are combined with one another in a manner specific to the application in order to meet a wide variety of requirements, has proven itself as a particularly simple and reproducible system for the processing of sample media.

### BRIEF DESCRIPTION OF THE FIGURES

FIG. 1A shows a 3D view of a system according to the invention with a mixing cylinder (2) with film-hinge lid, two separating units or separating devices (3), a flow-regulating connection unit (5), which is formed as a vacuum unit (5), and a collecting vessel (7), which are plugged into one another.

FIG. 1B shows a side view of the system according to FIG. 1A.

FIG. 1C shows a sectional side view of the system according to FIG. 1A.

FIG. 1D shows a view of the system according to FIG. 1A from above.

FIG. 1E shows a view of the system according to FIG. 1A from below.

FIG. 2A shows a side view of the mixing cylinder (2) with opened lid.

FIG. 2B shows a side view of the mixing cylinder (2) with closed lid.

FIG. 2C shows a rear view of the mixing cylinder (2) with closed lid.

FIG. 2D shows a sectional view of the mixing cylinder (2) with opened lid.

FIG. 2E shows a sectional view of the mixing cylinder (2) with closed lid.

FIG. 2F shows a view of the mixing cylinder (2) with opened lid from below.

FIG. 2G shows a view of the mixing cylinder (2) with opened lid from above.

FIG. 2H shows a view of the mixing cylinder (2) with opened lid from below.

FIG. 2I shows a view of the mixing cylinder (2) with opened lid from below.

FIG. 3A shows a 3D view of the separating device (3).

FIG. 3B shows a side view of the separating device (3).

FIG. 3C shows a front view of the separating device (3).

FIG. 3D shows a sectional view of the separating device (3).

FIG. 3E shows a view of the separating device (3) from above.

FIG. 3F shows a view of the separating device (3) from below.

FIG. 3G shows a linear seal variant of the separating device (3).

FIG. 3H shows another linear seal variant of the separating device (3).

FIG. 3I shows a union of two separating units (3), where preferably the upper of the two separating units (3) has a filter or a separating membrane with a pore diameter x, and the lower of the two separating units (3) has a filter of a separating membrane with a pore diameter <x, such that a sieve cascade is produced.

FIG. 3K shows a sectional view of the two separating units (3) with variants of the linear seal.

FIG. 3L shows a view of a separating unit (3) with a backwash plane, as a result of which a directed backwash is permitted.

FIG. 3M shows a side view of a separating unit (3) (with sieve) with backwash device for the backwash of the retentate after use of the system according to the invention, and after the separating unit (3) has been detached from the other functional units of the modular system and has been connected to another collecting vessel (6), e.g. a centrifuge tube (6).

FIG. 3N shows a side view of the arrangement according to FIG. 3M.

FIG. 3O shows a sectional view of the arrangement according to FIG. 3M.

FIG. 4A shows a 3D view of the vacuum unit (5).

FIG. 4B shows a side view of the vacuum unit (5).

FIG. 4C shows a sectional view of the vacuum unit (5).

FIG. 4D shows a plan view of the vacuum unit (5).

FIG. 4E shows a system according to the invention with attachment of a vacuum device—before the filtration.

FIG. 4F shows a system according to the invention with attachment of a vacuum device—after the filtration.

FIG. 4G shows a structure of a system according to the invention for releasing cells on the sieve surface of the separating unit (3).

FIG. 4H shows a sectional view of a structure for releasing cells from the membrane surface of the separating unit (3), in particular of the structure according to FIG. 4G.

FIG. 5A shows a schematic view of the closure of a mixing cylinder (2) with a base cap (8) or of a mixing cylinder (2) that can be closed with a base cap (8).

FIG. 5B shows a sectional view of the base cap fixation.

FIG. 5C shows a sectional view of the necessary geometry on the mixing cylinder for fixation of the base cap.

### LIST OF REFERENCE SIGNS

1 separating units
2 mixing cylinder
3 separating membrane
5 vacuum unit
7 collecting/receiving container
8 base cap
20 film hinge
21 peripheral edge
22 cutout for opening the lid
23 lid
24 outer wall of the mixing cylinder
25 surface fit
26 linear seal
27 inner wall of the mixing cylinder
28 closure foil
29 tear-off tab for the closure foil
30 outer wall of a separating unit above the grip piece
31 grip piece
32 spacer
33 webs
34 outer wall of a separating unit below the grip piece
35 surface fit
36 linear seal
37 cutout for ventilation on the outer wall of the separating unit below the grip piece
38 cutout for ventilation on the grip piece
39 separating membrane
40 backwash plane
41 backwash reservoir
42 outflow edge
50 outer wall of the vacuum unit
51 connector piece, male
52 surface fit vacuum of the vacuum unit
53 linear seal
54 lugs of the connector piece
55 starting solution
56 nonreturn valve
57 disposable syringe
58 filtrate
59 reservoir
60 interspace for preventing liquid aspiration with the vacuum device
80 outer geometry of the base cap
81 inner geometry of the base cap
82 clamp area 1
83 clamp area 2
84 clamp area 3
85 spacer
86 geometry of the mixing cylinder for fixing the base cap

All of the features set out in the above description and in the attached claims and figures can be used both singly and also in any desired combination to implement the invention in the various embodiments thereof.

### DETAILED DESCRIPTION OF THE INVENTION

The invention accordingly relates to a system of freely combinable functional units, which system is suitable in particular for mixing, processing and/or separating samples and consists of
at least one mixing cylinder (2),
at least one separating device (3) and
a flow-regulating connection unit (5), which flow-regulating
connection unit (5) is preferably formed as a vacuum unit,
   and which system is designed such that the functional units (2; 3; 5) are connected, preferably via simple plug connections, in any desired sequence, in particular to as many functional units as necessary, in order to form a preferably inherently closed sample-processing system.

The functional units according to the invention therefore preferably consist of or are
mixing cylinder(s),
separating device(s) and
a vacuum unit.

The system permits the use of different cylindrical containers (closed at one end), for example test tubes. This is achieved by scaling of the dimensions. The cylindrical containers can be used as collecting or receiving vessels for the supernatants or the respective fractions.

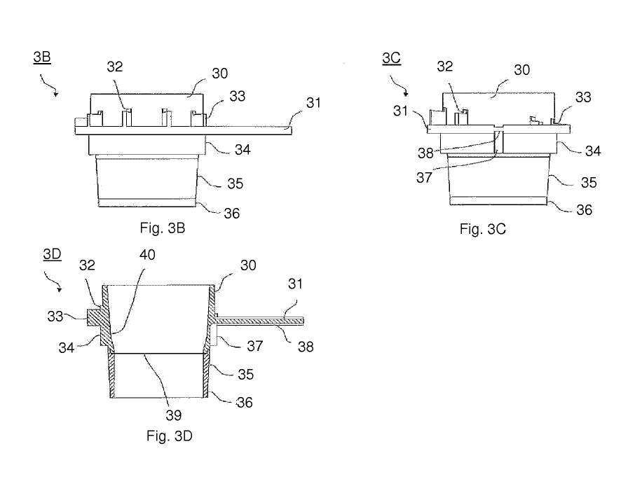

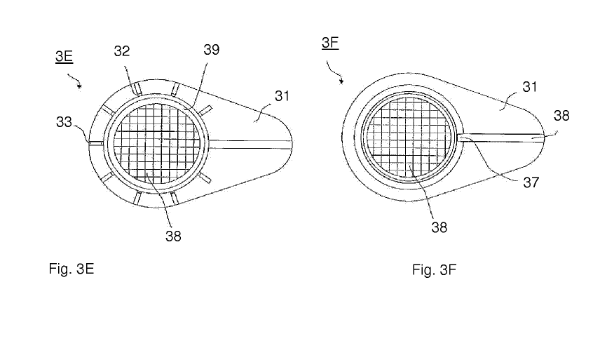

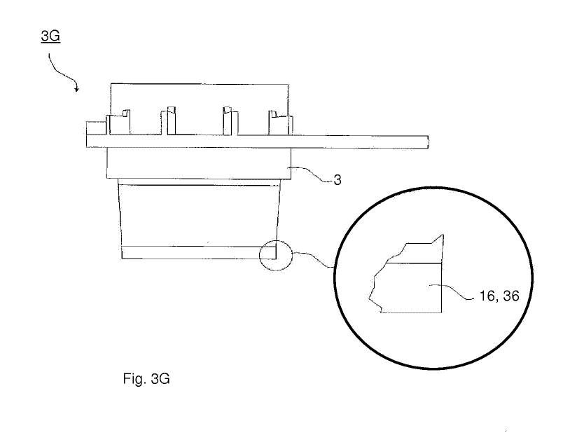

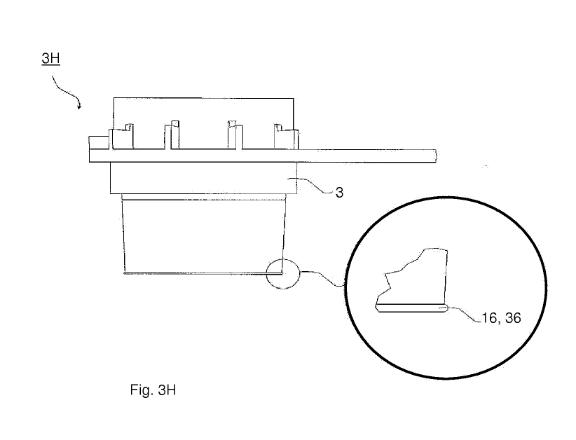

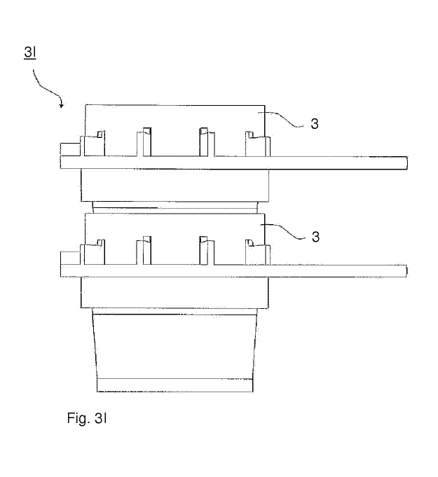

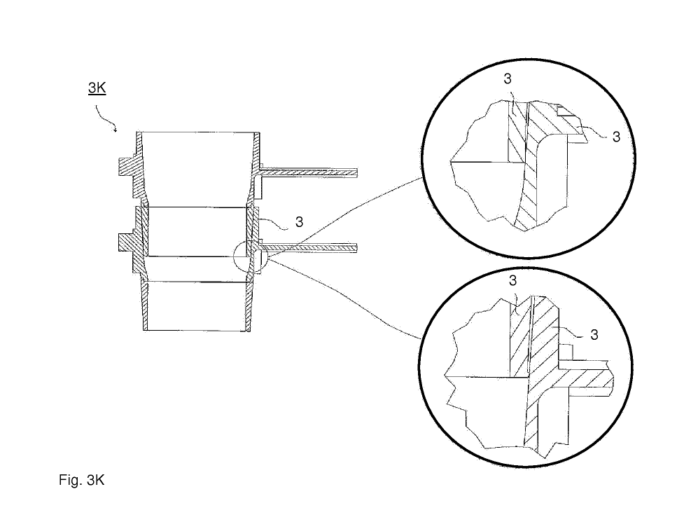

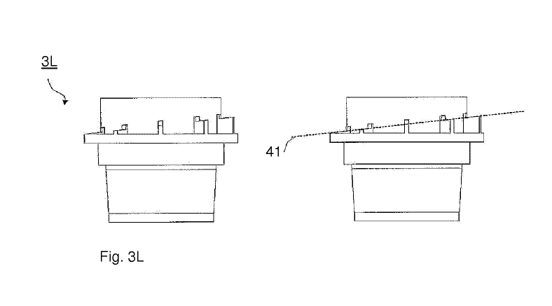

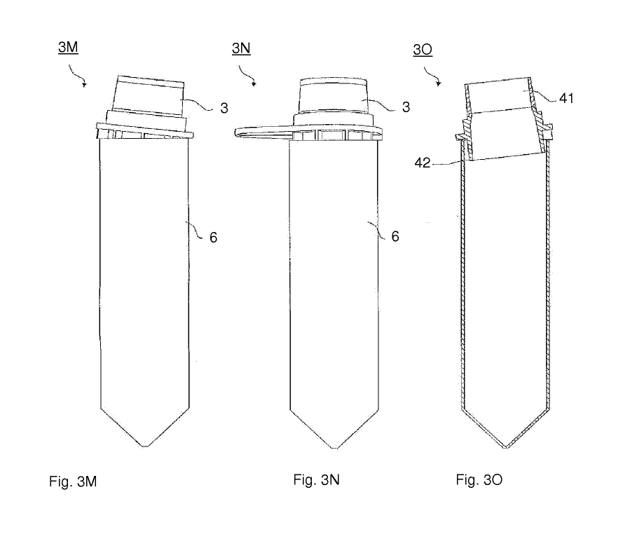

An important criterion proves to be the configuration of the transitions between the preferably plugged-together functional units. This connection is preferably achieved by a wedge-shaped geometry. In order to ensure that sample material is not drawn by capillary forces into the sealing space between the functional units, at least one seal, in particular a linear seal, preferably (made of) propylene, is used. A number of variants are possible for the sealing (see, for example, FIGS. 3G and 3H). Variant one is straight in the lower area of the wedge, such that a circumferential connection is obtained between two functional units. Advantageously, variant one has a geometry that can be more easily produced by injection moulding. Variant two is a circumferential ring at the end of the plug connection, which is preferably attached directly to the wedge connection by injection moulding. This leads to an additional ring connection. There is also the possibility of generating this seal as an O-ring or by two-component injection moulding. An inclined plane on the top face of the separating units (backwash plane) permits a directed backwash of the retentate from the filter surface with simultaneous reduction of the amount of backwash liquid. The backwash plane according to the invention is preferably an arrangement of a plurality of webs, preferably 6-18 webs, which are arranged on the outside of the separating unit (3) and/or protrude from the separating unit (3), said webs being formed and arranged in such a way that the underside of the webs ends on an (imaginary) contact plane inclined with respect to the top and/or underside of the separating unit, or the underside of the webs forms an (imaginary) contact plane inclined with respect to the top and/or underside of the separating unit (3) when the separating unit (3) is placed onto a receiving container (7). The incline of the (imaginary) plane is preferably formed at an angle of 2-45°, preferably 5-40°.

Spacers (32) preferably ensure that, during the backwash procedure, the separating device (3) does not come into contact with the wall of the receiving container (7), which is preferably formed as a test tube.

The individual constituents or functional units of the modular system according to the invention are explained in more detail below.

1. Mixing Cylinder

The mixing cylinder is preferably open at both ends, although it can also be open just at one end (such that the sample material would be tipped out onto the separating device), and is closed at one end by a lid, which preferably has a film hinge, or by a lid designed as a screw-on lid, stopper or plug-on cap, and is closed at the other end by a tear-off and/or pierceable foil and/or a by base cap that can be plugged on, inserted like a stopper or screwed on.

The mixing cylinder is used to receive the sample and can be used as an alternative to test tubes open at one end. The interior is designed such that it can serve simultaneously as a mixing vessel for obtaining biological, chemical or physical reactions. The geometry of the mixing cylinder ensures
low shearing forces, particularly in incubation with biological material such as primary cells, cell lines, etc.
minimal dead spaces
maximum sample recovery
   and is preferably suitable for incubation on a roller mixer and for incubation on an overhead mixer.

Preferred features for meeting the requirements are:

Both ends of the mixing cylinder have a different geometry:
Filling end (but can also be taken for tilting the liquid out onto the separating device):
The filling end is closed by a lid with film hinge (for other closure possibilities, see above). From here, the mixing cylinder is filled with sample material and optionally reaction material and solutions. The film hinge permits aseptic working. For the use of the mixing cylinder in combination with mechanical mixing devices, the lid has a circumferential edge. The circumferential edge avoids the cylinder catching or becoming stuck during the mixing procedure (in particular on a tilting roller mixer/roller mixer); in order to ensure an unimpeded rotation of the mixing cylinder with film hinge, the lid is preferably additionally modified with a circumferential edge.
Emptying end (can also be used as filling end):
The emptying end of the mixing cylinder is constructed such that it can be plugged or inserted like a stopper into the separating device or can be screwed onto the latter. The emptying end can optionally be closed with a foil and/or base cap (see above: preferably by plugging, stoppering or screwing). The foil (disposable after one use) is secured to the cylinder by heat (thermally sealed). The foil is removed when necessary (e.g. after incubation with substances) by being torn off by the applied projection or by being perforated by a pointed article, such as a pipette tip, or by a piercing device directly on the separating device. The use of a base cap permits aseptic working. The whole fitting area/insertion area of the mixing cylinder is closed by the base cap. By use of the base cap, the emptying end of the mixing cylinder can at the same time be used as filling end. The emptying end can optionally be equipped directly with a sieve which, for example, permits the preliminary separation of desired fractions or the segregation of undesired fractions. Desired fractions that are retained can be used directly or, by reclosing the mixing cylinder with the base cap, can be used for further biological, chemical or physical reactions. By virtue of the design of the cylinder, the latter, upon combination with a sieve on the emptying end, can be combined directly with the flow-regulating connection unit and can thereby be used like the separating device. Undesired fractions that are retained increase the purity of the following separation with the separating device.

Both ends of the mixing cylinder are preferably of the same design:

Each end can be used as filling and/or emptying end, the filling and/or emptying end being designed with a lid, in particular with one of the abovementioned lids, e.g. with a removable lid. The direction of combination with a separating device can be chosen freely. Each of the ends can be connected to the separating device.

2. Separating Device

The separating devices are based on the filtering principle. The filter material and the pore size are provided in accordance with the problem to be solved. It can serve both for separation by size and also for the immobilisation of substances. Since the separating devices themselves serve as vessels, they can also serve as reaction space for chemical reactions (e.g. activation and/or coupling of proteins) and also receive substances with selective properties (e.g. with defined affinities).

3. Flow-Regulating Connection Unit (Adapter), which is Preferably Formed as a Vacuum Unit.

Vacuum unit within the meaning of the invention is understood in particular as a transition piece with (laterally arranged) suction connector, preferably for connection to a vacuum pump, such that a suction bottle is produced by connecting the vacuum unit (5) to the separating vessel (7), for example by placing or screwing/clamping the vacuum unit (5) onto the separating vessel (7).

The flow-regulating connection unit permits the connection of the separating device to different cylindrical containers (closed at one end), for example test tubes, and serves as adapter. The shape is accordingly adapted at one end to the geometry of the separating device and at the opposite end to the geometry of the respective cylindrical containers. The connection unit is responsible for a defined flow of liquid and supports the separating unit in the use of at least one separating device, preferably more than two separating devices. By closing the connection unit, it is possible for liquid for incubation purposes (e.g. for immunochemical reactions) to be kept in the separating device. After the closure of the connection unit is opened, the liquid flows into the cylindrical container. Depending on the problem to be solved, the target substance can either be located in the through-flow, immobilised on the membrane or on solid supports that have been introduced into the reaction space of the separating device.

The connection unit can be connected to the collecting and/or waste vessel either by a thread or by a wedge construction.

Experience shows that test tubes differ greatly in thread pitch depending on the manufacturer, but the internal diameter of the test tubes varies only by a few micrometers. This can preferably be compensated by a simple wedge construction of the vacuum unit. The fixing takes place preferably as an “interference fit”. This results in a system that gives the user the possibility of using existing test tubes.

The connection unit permits the combination with a vacuum filtration by attachment of underpressure-generating devices and device combinations (for example disposable syringes, hose pump, vacuum pump, water jet pump, pipette with hose, and many other devices that can generate underpressure, vacuum).

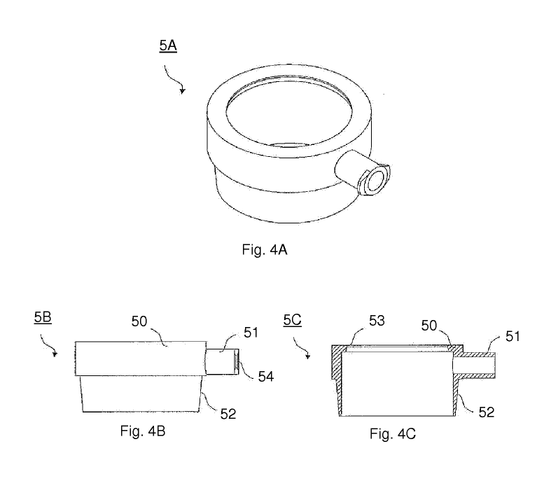

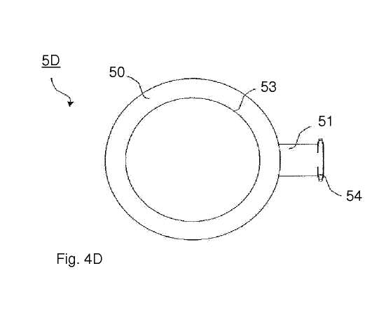

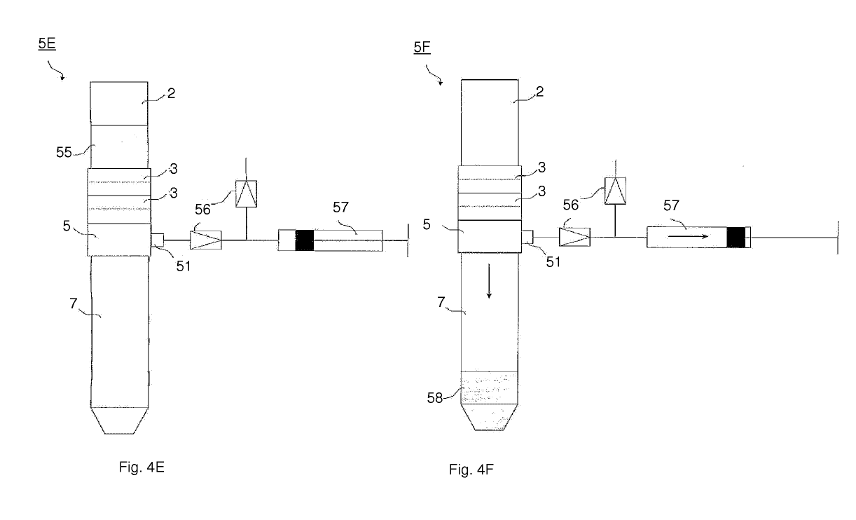

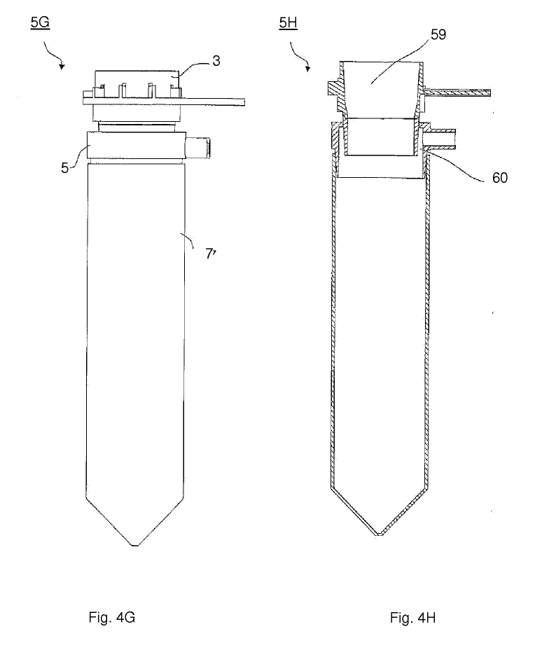

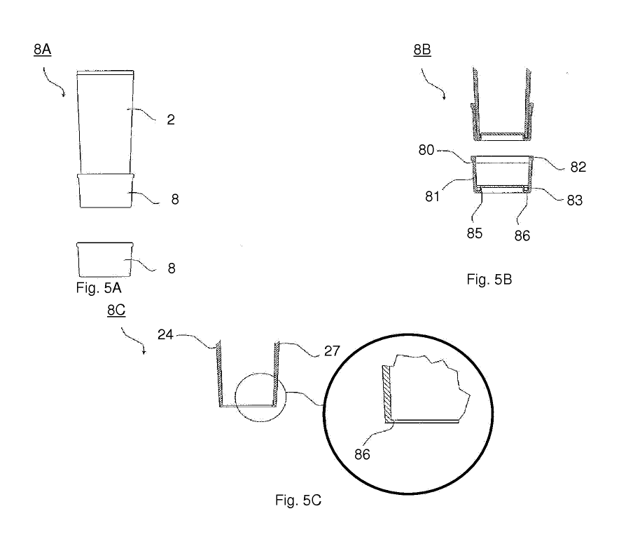

The principle of vacuum filtration shown in FIGS. 4E and 4F is explained briefly below and serves as an example for the use of underpressure-generating devices and device combinations.

The vacuum unit was constructed such that a movement of liquid takes place exclusively via the movement of the disposable syringe (57). Since the system is closed, no liquid can run from the mixing cylinder (2) via the separating unit (3) into the receiving vessel (7). The used membrane material serves as additional barrier and prevents the exchange of air from the receiving vessel by the liquid in the recipient. When the syringe is drawn up, an underpressure forms in the receiving vessel and conveys the liquid from the recipient through the separating membrane into the receiving vessel. The two nonreturn valves (56) allow the air to be displaced from the syringe without air being forced back into the separating units.

Other advantageous features and configurations of the system according to the invention will also become clear from the figures, which show various embodiments of the modular system and of the functional units of the latter.

The system of freely combinable functional units according to the invention consisting of
at least one mixing cylinder (2), preferably one mixing cylinder (2),
at least one separating device (3), preferably two to eight separating devices (3) and
a flow-regulating connection unit, which is preferably formed as vacuum unit (5),
   is designed such that the functional units are connected, preferably via plug connections, in any desired sequence, in particular to as many functional units as necessary, in order to form a preferably inherently closed sample-processing system, in particular for mixing, processing and/or separating samples.

The system is preferably formed, in particular in terms of the dimensions of its functional units, such that it can be applied or connected to commercially available receptacles, in particular laboratory tubes, preferably with a volume of 5-100 ml, preferably a filling volume of 15 ml or 50 ml.

In a preferred embodiment, the system according to the invention is characterised in that the mixing cylinder (2) can be temporarily closed at both ends from both sides.

In another preferred embodiment of the system, the interior of the mixing cylinder(s) is designed such that an optimal and thorough mixing of the samples is permitted.

In another advantageous embodiment of the invention, the system is designed such that the one or more separating devices are based on the filtering principle, i.e. in particular that the one or more separating devices each have a filter, a membrane and/or a sieve or are designed with these. The filters, membranes and/or sieves preferably have pores with a pore diameter of between 10 nm and 1 mm.

In another preferred embodiment, the at least one separating device is designed such that, for substance separation, membranes can be used that have a pore diameter of between 10 nm and 1 mm.

In a particularly preferred embodiment of the system according to the invention, the one or more membranes are made of one more materials known per se and can be used both as size filters and/or also as solid supports with an immobilising surface configuration.

In another particularly preferred embodiment, the system according to the invention is characterised in that the at least one separating device can be used as reaction vessel for chemical and/or physical reactions.

Another advantageous embodiment concerns a system according to the invention in which the at least one separating device can be provided if necessary with openings for charging or emptying.

Another advantageous embodiment concerns a system according to the invention in which a flow-regulating connection unit with three-way tap forms the closure of the sample-processing system.

In a particularly advantageous development of the system according to the invention, the dwell time/transit time of the sample in/through the functional units can be freely regulated by the flow-regulating connection unit.

Another advantageous embodiment of the system according to the invention is characterised in that it can be connected to commercially available vessels for receiving the through-flow.

A system according to the invention is particularly preferred that consists of
a mixing cylinder (2),
two to eight, preferably two to five, particularly preferably two or three, separating devices (3), and
a flow-regulating connection unit, in particular a vacuum unit (5),
   which are arranged in series in this sequence, where the two to eight separating devices (3) are preferably designed as sieve cascades by suitable choice of the pore size, i.e. the pore diameter of the filter, membrane or sieve of the mixing device decreases from the top downwards, i.e. in the direction of the flow-regulating connection unit, such that, for example, microparticles of different size can be separated across this cascade sieve.

In particular, a system according to the invention is preferred in which the at least one mixing cylinder (2) has at least one of the features 20-29 and/or 80-86 or preferably a combination of such features or particularly preferably all the features 20-29 and/or 80-86 according to the list of reference signs and/or the figures.

Another preferred system according to the invention is characterised in that the at least one separating device (3) has at least one of the features 30-42 or preferably a combination of such features or particularly preferably all the features 30-42 according to the list of reference signs and/or the figures.

Another preferred system according to the invention is characterised in that the flow-regulating connection unit (5), which is preferably formed as a vacuum unit (5), has at least one of the features 50-60 or preferably a combination of such features or particularly preferably all the features 50-60 according to the list of reference signs and/or the figures.

Another preferred embodiment concerns a system according to the invention, in particular suitable as a separating device, which also has (as a further functional unit) a collecting and/or receiving container (7), which is preferably connected to the flow-regulating connection unit (preferably vacuum unit) (5).

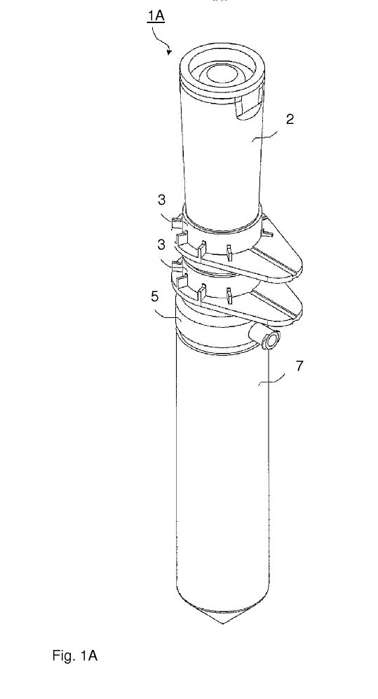

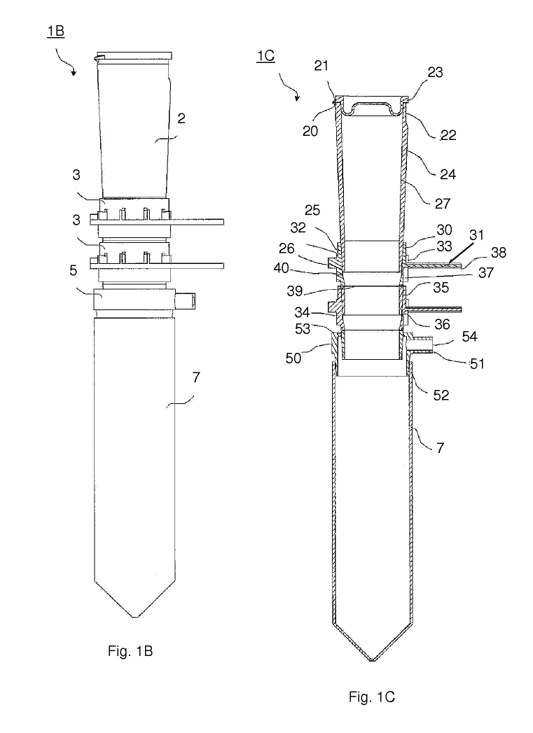

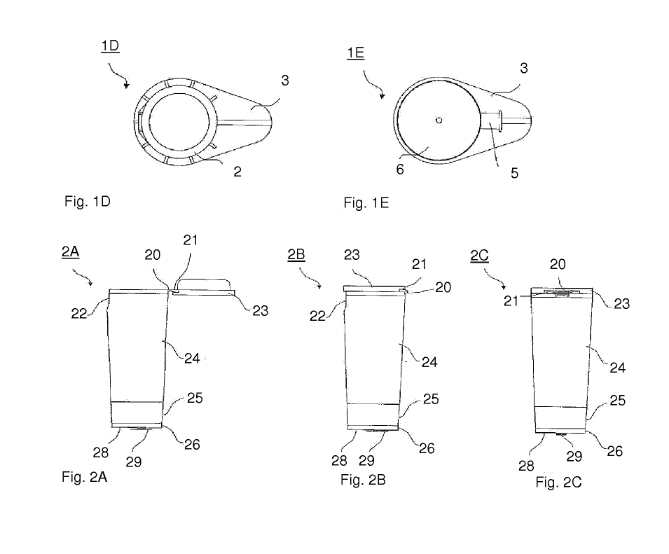

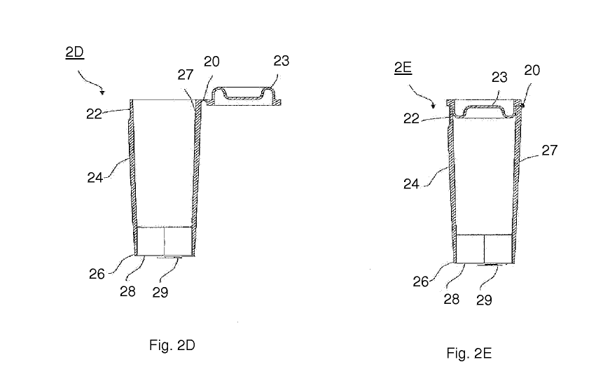

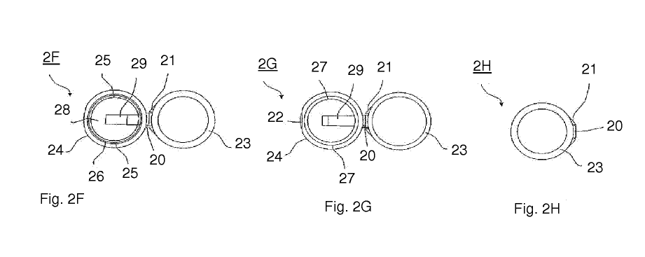

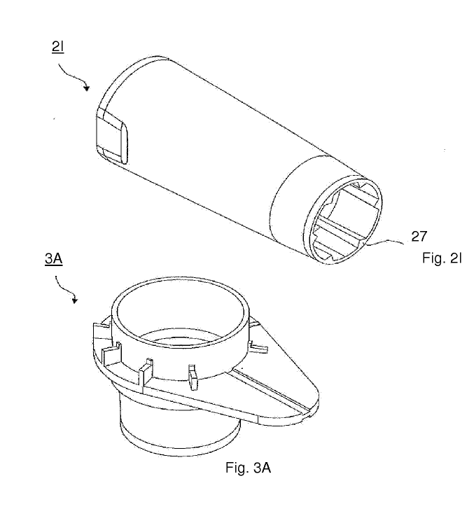

Another advantageous development of the invention concerns a system, in particular designed as a separating unit, which is designed according to FIGS. 1A-1E.

Another aspect of the invention relates to the use of the system according to the invention for application in biological and/or medical research, for diagnosis and/or for product control in biotechnology, in particular for mixing, processing and/or separating samples.

Other advantageous properties and features of the invention will also become clear from the following non-limiting illustrative embodiments.

### Application Example 1

Isolation of specific cells from a blood sample, explained using the example of separating CD3-positive cells from whole blood with subsequent
(A) isolation of RNA or DNA
(B) recovery of the individual cells
(C) backwash of the particle-cell complexes
(D) use of the specifically depleted sample.

A mixing cylinder, a separating device, a flow-regulating connection unit and two receiving containers are needed for this application.

Whole blood to which anticoagulants have been added is introduced with anti-CD3 particles into the mixing cylinder at the filling end. The mixing cylinder is closed and is rotated on a roller mixer for at least 10 minutes and at most 45 minutes. After the incubation, the mixing cylinder is opened at the emptying end. The separating device with a receiving container is then applied to the cylinder. The assembly of mixing cylinder, separating device and receiving vessel is turned. The mixing cylinder is then opened at the filling end. The mixture of whole blood and anti-CD3 particles then runs through the separating device. The particles that have captured the CD3-positive cells from the whole blood remain on the membrane of the separating device. Unbound and undesired cells are flushed into the receiving vessel by repeated flushing with a wash solution.
(A) The separating device together with the particle/cell complexes is then applied to a fresh test tube combined with a flow-regulating connection unit. The connection unit is closed by a cap, such that no exchange of air and liquid can take place through the separating device. A lysis liquid for releasing the RNA or DNA from the cells is then fed into the reaction space of the separating device and incubated. After the incubation, the cap of the connection unit is removed and replaced by a disposable syringe. The entire liquid together with the specific RNA from the CD3-positive cells is then transferred with the aid of the syringe into the receiving vessel. The particles remain on the membrane. The purification of the RNA or DNA from the cell lysate then takes place with the separation methods available in the prior art for RNA or DNA.
(B) The separating device together with the particle/cell complexes is then applied to a fresh test tube combined with a flow-regulating connection unit. The connection unit is closed by a cap, such that no exchange of air and liquid can take place through the separating device. A special release liquid for separating the cells from the particles is then fed into the reaction space of the separating device and incubated. After the incubation, the cap of the connection unit is removed. The cells are transferred into the receiving vessel by repeated flushing with a wash solution. The specific cells, CD3-positive cells, free of undesired cells, are then available to the user for further analyses.
(C) The separating device together with the particle/cell complexes is turned and applied to a fresh test tube. By flushing with a wash solution, the particle/cell complexes are flushed into the fresh test tube. This fraction can then be used, for example, for culturing the cells directly on the particle.
(D) The undesired cells, which can be flushed into the receiving vessel by repeated flushing with a wash solution, are used as depleted fraction for further analyses.

### Application Example 2

Cascade application for simultaneous isolation of different specific fractions from a blood sample, explained using the example of separating CD4-positive and CD8-positive cells from whole blood, as rapid test for determining HIV immune status.

A mixing cylinder, two separating devices, two flow-regulating connection units and at least three receiving containers are needed for this application.

Two different particle sizes are needed in accordance with this application:
small particles that capture the CD4-positive cells
medium-sized particles that capture the CD8-positive cells.

In accordance with the two particle sizes, two separating devices with different pore sizes are needed:
small pore size for retention of the small particles
large pore size for retention of the large particles.

Whole blood to which anticoagulants have been added is introduced with the small anti-CD4 particles and the large anti-CD8 particles into the mixing cylinder at the filling end. The mixing cylinder is closed and is rotated on a roller mixer for at least 10 minutes and at most 45 minutes. After the incubation, the mixing cylinder is opened at the emptying end. The separating device with the large pore size is then applied first to the cylinder, and then the separating device with the small pore size. Lastly, the receiving container is positioned on the last separating device. The assembly of mixing cylinder, separating devices and receiving container is then turned and the mixing cylinder is opened at the filling end. The large CD8 particles together with the cells remain on the membrane of the first separating device, the smaller CD4 particles pass through the first separating device and remain on the lower separating device, and undesired and also unbound cells pass through the separating devices and are collected in the receiving vessel. Each of the separating devices together with the particle/cell complexes can then be used as an independent fraction. To determine the HIV immune status, a ratio of the isolated CD4-positive cells to CD8-positive cells is formed. Points (A)-(D) from application example one can likewise be applied for each of the fractions.

### Application Example 3

Cascade application for simultaneous isolation of different cytokines for the specific determination of the inflammation status from a blood sample.

A mixing cylinder, three separating devices, three flow-regulating connection units and at least four receiving containers are needed for this application.

Three different particle sizes are needed in accordance with this application:
small particles that capture IL-6
medium-sized particles that capture IL-8
large particles that capture TNFalpha.

In accordance with the three particle sizes, three separating devices with different pore sizes are needed:
small pore size for retention of the small particles
medium pore size for retention of the medium-sized particles
large pore size for retention of the large particles.

The isolation of the specific cytokines takes place in the manner explained in application examples one and two. Each of the separated fractions is then incubated with chemicals on the corresponding separating unit in such a way that the loading of the particles with the respective protein can be detected by a colour change. The intensity of the colour change in the reaction charge can be determined with the aid of a simple measuring device. With the measurement values obtained, a corresponding diagnosis can then be made regarding the inflammation status.

1.-21. (canceled)

22. A system of freely combinable functional units comprising:
at least one mixing cylinder,
at least one separating device and
a flow-regulating connection unit,
designed such that the functional units are connected, to form an inherently closed sample-processing system, for at least one of mixing, processing and separating samples, in which the mixing cylinder can be temporarily closed at both ends from both sides.

23. The system of claim 22, wherein the interior of the mixing cylinders permits an optimal and thorough mixing of the samples.

24. The system of claim 22, wherein the separating devices are filters.

25. The system of claim 22, wherein said separating device comprises a membrane with a pore diameter of between 10 nm and 1 mm.

26. The system of claim 25, wherein the membrane is a size filter and a solid supports with an immobilising surface configuration.

27. The system of claim 22, wherein the separating device is a reaction vessel for chemical and/or physical reactions.

28. The system of claim 22, wherein the separating devices are provided with openings for charging or emptying.

29. The system of claim 22, wherein the sample-processing system is closed by a flow-regulating connection unit with three-way tap.

30. The system of claim 28, wherein a dwell time of a sample in a functional unit is regulated by the flow-regulating connection unit.

31. The system of claim 22, further comprising connected vessels for receiving through-flow.

32. The system of claim 22, consisting of
a mixing cylinder,
one to five separating devices, and
a flow-regulating connection unit, which is a vacuum unit,
which are arranged in series in the given sequence.

33. The system of claim 22, wherein the at least one mixing cylinder comprises a lid, a film hinge, an outer wall, a surface fit, a linear seal, a closure foil, and a tear-off tab for the closure foil.

34. The system of claim 22, wherein the at least one separating device comprises a grip piece, spacer, web, surface fit, linear seal, and ventilation cut out.

35. The system of claim 22, wherein the flow-regulating connection unit is a vacuum unit, and comprises a linear seal, nonreturn valve, disposable syringe, filtrate reservoir, and interspace for preventing liquid aspiration within the vacuum unit.

36. The system of claim 22 designed as a separating unit, further comprising a collecting and receiving container, said collecting and receiving container connected to the flow-regulating connection unit.

37. The system of claim 36, designed as a separating unit, comprising a mixing cylinder comprising a lid with film hinge, two separating units respectively separating devices, a flow-regulating connection unit, which is formed as a vacuum unit, and a receiving container, all of which are nested.

38. A method for separating, mixing or processing a sample, comprising:
(a) providing a sample;
(b) introducing the sample into the system of claim 1;
(c) operating the system of claim 1 until the sample is separated, mixed, or processed.

39. The method of claim 38, wherein said sample is a biological research sample, medical research sample, biotechnology diagnosis sample, or biotechnology product control sample.

40. The method of claim 38, said system comprising a membrane having a pore diameter between 10 nm and 1 mm, wherein said membrane is a size filter and solid support with an immobilizing surface configuration.

41. The method of claim 38, wherein said processing includes chemical and/or physical reaction in said at least one separating device.
